(19) United States
(12) Patent Application Publication
(10) Pub. No.: US 2012/0059013 A1
(43) Pub. Date: Mar. 8, 2012

(54) METHOD OF TREATING EARLY MORNING AKINESIA IN SUBJECTS HAVING PARKINSON'S DISEASE

(75) Inventors: Boris FERGER, Biberach (DE); Erich BUERGER, Bingen am Rhein (DE); Makoto SHIMASAKI, Biberach (DE)

(73) Assignee: BOEHRINGER INGELHEIM INTERNATIONAL GMBH, Ingelheim am Rhein (DE)

(21) Appl. No.: 13/041,880

(22) Filed: Mar. 7, 2011

Related U.S. Application Data
(60) Provisional application No. 61/311,905, filed on Mar. 9, 2010.

(51) Int. Cl. A61K 31/428; A61P 25/14; A61K 31/506; A61K 31/496; A61K 31/4745

(52) U.S. Cl. 514/252.2; 514/367; 514/254.02; 514/292

(57) ABSTRACT

The present invention provides a method for treating early morning akinesia, comprising continuous administration to a patient in need of such treatment a therapeutically effective amount of a dopamine agonist or a pharmaceutically acceptable salt, enantiomer, solvate, hydrate, polymorph or prodrug thereof.

### BACKGROUND OF THE INVENTION

The present invention is related to a method of treating early morning akinesia in subjects having Parkinson's Disease.

Akinesia is the inability to initiate movement due to difficulty selecting and/or activating motor programs in the central nervous system. Common in severe cases of Parkinson's Disease, akinesia is a result of severely diminished dopaminergic cell activity in the direct pathway of movement.

Dopaminergic cell activity is related to the endogenous dopamine receptor stimulation by its natural ligand dopamine and by exogenously applied dopamine derived from levodopa or by synthetic dopamine receptor agonists. In Parkinson's Disease the treatment of the impairment of dopaminergic activity is complicated by short half-life of levodopa and well tolerated dopamine receptor agonists which results in nocturnal akinesia associated with sleep disturbances and early morning akinesia.

A dopamine agonist is a compound that binds to one or more of the different types and subtypes of dopamine receptors and stimulates neural signaling via the dopaminergic system. Dopamine agonists activate dopamine receptors in the absence of the dopamine ligand, and activate signaling pathways through the dopamine receptor and trimeric G-proteins ultimately leading to changes in gene transcription. Dopamine agonists are typically used for treating Parkinson's Disease and certain pituitary tumors, and may be useful for restless legs syndrome (RLS). There are a variety of methods used for the treatment of Parkinson's Disease and related disorders of early morning akinesia. Typically, these methods are involved in the administration of dopamine agonists to a patient in need several times a day, which result in fluctuating plasma and brain levels of the dopamine agonists. As a result, these methods are either ineffective in the treatment, or cause undesired side effects, for example, weight loss, sleep disturbances cardiac effects, or blood pressure effects; and/or have a delayed onset of action.

In view of the foregoing, there remains a need for new methods for the treatment of early morning akinesia in patients having Parkinson's Disease, including the reduction or elimination of one or more symptoms, diseases or conditions associated with or resulting from early morning akinesia.

### SUMMARY OF THE INVENTION

It is therefore an object of the present invention to provide new methods for the treatment of early morning akinesia, including the alleviation or elimination of one or more symptoms, diseases or conditions associated with or resulting from early morning akinesia. The invention achieves these objects and satisfies additional objects and advantages by providing methods of treating early morning akinesia, which comprises continuous administration to a patient in need of such treatment of a therapeutically effective amount of a dopamine agonist or a pharmaceutically acceptable salt, enantiomer, solvate, hydrate, polymorph or prodrug thereof.

In one embodiment, the present invention provides methods for the treatment of early morning akinesia comprising continuously administering a dopamine agonist to a patient in need via subcutaneous infusion of the dopamine agonist.

In another embodiment, the present invention provides methods for the treatment of early morning akinesia comprising continuous administration of a dopamine agonist to a patient in need via oral sustained or extended release formulations where the dopamine agonist is an active ingredient.

In an additional embodiment, the present invention provides methods for the treatment of early morning akinesia comprising continuous administration of a dopamine agonist to a patient in need thereof wherein the dopamine agonist is pramipexole or a pharmaceutically acceptable salt thereof.

The foregoing objects and additional objects, features, aspects and advantages of the present invention are further exemplified and described in the following detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A illustrates a comparative animal study of effects among continuous release of pramipexole, immediate release of pramipexole and vehicle on the time course of haloperidol-induced catalepsy.

FIG. 1B illustrates a comparative animal study of effects among continuous release of pramipexole, immediate release of pramipexole and vehicle on the cumulative data of haloperidol-induced catalepsy.

FIG. 2A illustrates a comparative animal study of effects among continuous release of pramipexole, immediate release of pramipexole and vehicle on the time course of reserpine-induced akinesia.

FIG. 2B illustrates a comparative animal study of effects among continuous release of pramipexole, immediate release of pramipexole and vehicle on the cumulative data of reserpine-induced akinesia.

FIG. 3A is an in vivo microdialysis animal study showing effects of continuous release of pramipexole and immediate release of pramipexole on extracellular levels of dopamine.

FIG. 3B is an in vivo microdialysis animal study showing effects of continuous release of pramipexole and immediate release of pramipexole on extracellular levels of pramipexole in the striatum.

FIG. 4 shows a blood plasma level of pramipexole over 24 hours in healthy adult men in fasted state that is built up and maintained by the once daily application of an extended release formulation wherein pramipexole is the active ingredient.

### DETAILED DESCRIPTION OF THE INVENTION

### Definitions

The term “treating” as used herein, unless otherwise indicated, means reversing, alleviating, inhibiting the progress of, or preventing, either partially or completely, early morning akinesia in a patient. The term “treatment” as used herein, unless otherwise indicated, refers to the act of treating.

The term “therapeutically effective amount” or “effective amount” means the amount of the subject compound or combination that will elicit the biological or medical response of a tissue, system, animal or human that is being sought by the researcher, veterinarian, medical doctor or other clinician.

The term “administered” or “administering” as used herein is meant parenteral and/or oral administration. By “parenteral” is meant intravenous, subcutaneous and intramuscular administration.

The term “pharmaceutically acceptable salts” refers to salts prepared from pharmaceutically acceptable non-toxic bases or acids. The non-toxic bases include inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Salts derived from such organic non-toxic bases include salts of primary, secondary, and tertiary amines, as well as cyclic amines and substituted amines such as naturally occurring and synthesized substituted amines. The non-toxic acids include inorganic and organic acids, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

The term “enantiomer” or “enantiomeric” refers to a molecule that is nonsuperimposeable on its minor image and hence optically active wherein the enantiomer rotates the plane of polarized light in one direction and its minor image rotates the plane of polarized light in the opposite direction.

The term “subject” refers to a mammalian subject and most preferably a human subject or human patient.

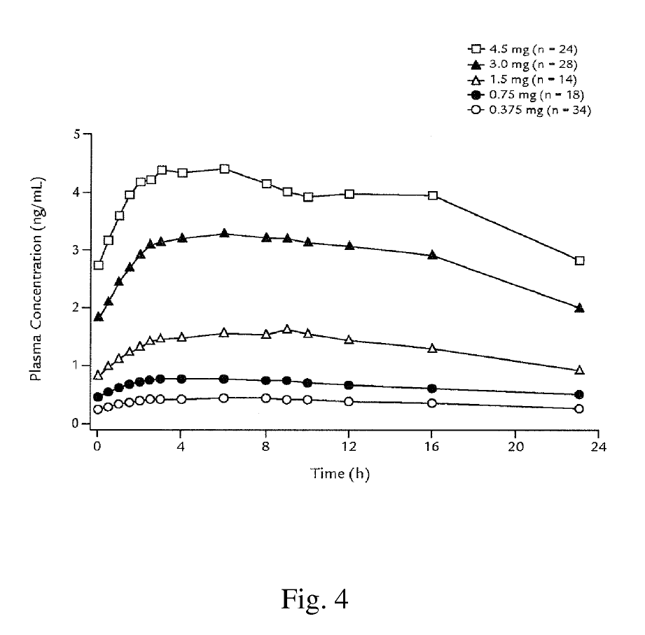

The term “continuous administration” refers to a continuous delivery of the dopamine agonist into the body of the patient over time in order to maintain a blood plasma level of the dopamine agonist that is substantially uniformly constant over the whole period of therapy. Non-limiting examples of the whole period of therapy include 12 hours, 24 hours, 36 hours, 48 hours 60 hours and 72 hours. The preferred whole period of therapy is 24 hours, so that the therapy may be applied on a daily basis. Over the time of therapy a substantially uniformly constant blood level can be maintained by the substantially continuous delivery of the active ingredient that is in about the same magnitude as the excretion thereof plus the metabolic transformation thereof into an inactive compound. FIG. 4 shows an example of this for the active ingredient pramipexole that is delivered by an orally administered extended release tablet for humans that provides a substantially constant blood plasma level of pramipexole for about 24 hours. This substantially constant blood plasma level can be further maintained over an extended period of time by repeated administration of the extended release tablet once daily, i.e. about once every 24 hours. This example makes it evident that the continuous delivery does not only include a continuous delivery of the active ingredient into the blood from the single administration of a single sustained release dosage form, but it also includes the evenly repeated administration of subsequent sustained release dosage forms in order to counteract the decrease in effect that may result from the elimination process with respect to the corresponding preceding dosage form.

The term “early mornig akinesia” refers to akinesia which is diagnosed in a patient up to 60 min, preferentially up to 30 min, after the patient wakes up in the morning.

### DESCRIPTION

The present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof.

In one embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the continuous administration of a dopamine agonist has an effect of continuous dopaminergic stimulation (“CDS”). CDS is desirable to avoid the non-physiological pulsatile dopamine receptor stimulation. CDS has an effect on prolonged therapeutic efficacy and results in: (1) lower propensity to develop motor fluctuations, dyskinesia and fewer nocturnal disturbances as well as early morning akinesia; and (2) prevention of unwanted effects related to fluctuations in brain and plasma drug levels.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the continuous administration of the dopamine agonist is achieved by subcutaneous infusion of a dopamine agonist. The subcutaneous infusion of the dopamine agonist can be achieved by one or more subcutaneously implanted minipumps.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where continuous administration of the dopamine agonist is achieved by an orally administered sustained or extended release formulation where the dopamine agonist is an active ingredient.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the patient has Parkinson's Disease.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the dopamine agonist is a nonergot dopamine agonist. Non-limiting examples of nonergot dopamine agonists include pramipexole, ropinirole, rotigotine, pardoprunox, sumanirole, apomorphine and piribedil.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the dopamine agonist is pramipexole (“PPX”) or a pharmaceutically acceptable salt thereof.

Pramipexole is commercially available and sold under the trademark Mirapex™ as the dihydrochloride monohydrate in treatment of early and/or advanced stages of Parkinson's Disease. Pramipexole can also be used for treatment of patients suffering from restless leg syndrome. Pramipexole can be used in monotherapy, as well as in combination with other anti-parkinsonian medication such as L-3,4-dihydroxyphenylalanine (“L-DOPA”) for the treatment of Parkinson's Disease. The chemical name of Pramipexole is (S)-2-amino-4,5,6,7,-tetrahydro-6-(propylamino)benzothiazole and the salt form commonly used is pramipexole dihydrochloride monohydrate. For the purpose of the present application, the term “Pramipexole” as used herein is considered to include pramipexole and any of its pharmaceutically acceptable salts. It is available as tablets for oral administration containing 0.125 mg, 0.25 mg, 0.5 mg, 0.75 mg, 1.0 mg and 1.5 mg of the active compound (calculated as free base). The tablets also contain various inactive ingredients including mannitol, corn starch, collodial silicon dioxide, povidone and magnesium stearate. Methods for the preparation of pramipexole and related compounds, methods for treatment and pharmaceutical compositions are known in the art. Thus, for example, U.S. Pat. No. 4,886,812 describes the compound pramipexole and pharmaceutically acceptable salts thereof, U.S. Pat. Nos. 6,001,861 and 6,194,445 related to a method of treating Restless Leg Syndrome (RLS) with pramipexole; and US Patent Application Publication Nos. 2006/0051419, 2005/0175691, 2007/0196481, 2006/0198887, 2005/0226926 and 2006/0051417 describe extended release pharmaceutical compositions comprising pramipexole or a pharmaceutically acceptable salt thereof. Such extended release formulations may comprise pramipexole, or a pharmaceutically acceptable salt thereof, in various therapeutically effective amounts, for example, in an amount of 0.375, 0.75, 1.5, 2.25, 3, 3.75 or 4.5 mg per daily dosage formulation.

It is preferred to apply the dopamine agonist in form of an extended release formulation which allows the dopamine agonist to build up a continuous blood plasma level of the active ingredient over the course of therapy. Preferably such formulation releases the active ingredient over a time of 24 hours, so that the formulation may be applied once daily.

Pramipexole is suitable for continuous dopaminergic stimulation because of its good tolerability and favorable pharmacokinetic properties in humans, for example, such as high oral bioavailablity, no significant interaction with hepatic cytochrome P450 enzymes, and long half-life. Pramipexole is a non-ergoline full dopamine receptor agonist, and has been shown to bind the D₂, D₃ and D₄ dopamine receptor subtypes with more selectivity for the D₃ receptor.

Preferred extended release formulations comprising Pramipexole are disclosed in US patent application Nos. 2006/0051419, 2006/0051417 and 2006/0198887, which are incorporated by reference.

A preferred formulation is, for example, disclosed in US patent application No. 2006/0198887, which is an extended release tablet which comprises one single, homogeneous matrix that comprises the full daily dosage of Pramipexole. Such a matrix preferably comprises at least one water-swellable anionic polymer or at least one water-swellable anionic polymer and one water-swellable neutral polymer.

Examples for such matrix tablets according to US patent application No. 2006/0198887 are given in Table 1.

TABLE 1

| | mg per | mg per | mg per | mg per | mg per | mg per | mg per |
|---|---|---|---|---|---|---|---|
| | 0.375 mg | 0.75 mg | 1.5 mg | 2.25 mg | 3.0 mg | 3.75 mg | 4.5 mg |
| Ingredient | tablet | tablet | tablet | tablet | tablet | tablet | tablet |
| Pramipexole | 0.375 | 0.750 | 1.500 | 2.250 | 3.000 | 3.750 | 4.500 |
| dihydrochloride | | | | | | | |
| monohydrate, | | | | | | | |
| Hypromellose | 112.500 | 148.500 | 157.500 | 180.000 | 191.250 | 202.500 | 225.000 |
| 2208 | | | | | | | |
| (Methocel | | | | | | | |
| K15M) | | | | | | | |
| Corn starch | 119.375 | 160.620 | 169.650 | 193.350 | 209.075 | 220.800 | 250.000 |
| Carbomer 941 | 15.000 | 16.500 | 17.500 | 20.000 | 17.000 | 18.000 | 15.000 |
| (Carbopol 71 | | | | | | | |
| G) | | | | | | | |
| Colloidal | 1.500 | 1.980 | 2.100 | 2.400 | 2.550 | 2.700 | 3.000 |
| silicon | | | | | | | |
| Dioxide | | | | | | | |
| Magnesium | 1.250 | 1.650 | 1.750 | 2.000 | 2.125 | 2.250 | 2.500 |
| Stearate | | | | | | | |
| Total | 250.000 | 330.000 | 350.000 | 400.000 | 425.000 | 450.000 | 500.000 |

According to the present invention, an appropriate oral sustained or extended release formulation comprising Pramipexole is administered once every 12 hours, once every 24 hours, once every 36 hours or once every 48 hours at a predetermined time of the day. Preferably, in the context of the present invention the oral sustained or extended release formulation comprising Pramipexole is administered once every 24 hours in the evening, before the patient goes to sleep. In this context the formulations according to table 1 are appropriate extended release types for a once daily administration.

The present invention demonstrates that continuous dopaminergic stimulation via continuous release of Pramipexole (“PPX-CR”) offers a higher therapeutic benefit to patient suffering early morning akinesia than immediate release of Pramipexole (“PPX-IR”).

Specifically, a study on continuous dopaminergic stimulation by continuous release of Pramipexole was conducted on two representative animal models. One of the animal models was Haloperidol-induced catalepsy, and the other was Reserpine-induced akinesia. The testing of Haloperidol-induced catalepsy showed that continuous release of Pramipexole by a dosage of, for example, 1 mg/kg/day, reversed the haloperidol-induced motor impairment in the morning and over the whole observation period of 12 hours. In contrast, immediate release of Pramipexole by a dosage of, for example, tid 1 mg/kg, treated on the day before the testing day, was not effective in the morning of the testing day. Further, immediate release of Pramipexole by a dosage of, for example, 1 mg/kg, provided on the testing day, only reduced catalepsy for 6 hours. The testing of Reserpine-induced akinesia showed that early morning akinesia indicated by the first motor activity measurement in the morning was significantly reversed by continuous release of Pramipexole by a dosage of, for example, 2 mg/kg/day. In comparison, immediate release of Pramipexole by a dosage of, for example, tid 0.3 mg/kg, treated on the day before the testing day, was not able to antagonize early morning akinesia.

These above-discussed results are in agreement with in vivo microdialysis measurements showing a sustained decrease of extracellular dopamine levels and a continuous Pramipexole exposure in the testing group of continuous release of Pramipexole. The phrase “sustained decrease of extracellular dopamine levels” as used herein means maintaining a level of extracellular dopamine which is lower than physiologic norms. In contrast, the testing group of immediate release of Pramipexole produced a transient decrease of extracellular dopamine levels over 6 hours and showed maximum Pramipexole levels 2 hours after dosing which decreased over the following 6 to 8 hours.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where such treatment reduces or eliminates one or more symptoms, diseases or conditions associated with or resulting from early morning akinesia. The symptom, disease or condition associated with or resulting from early morning akinesia includes, but is not limited to, catalepsy, muscle rigidity, and motor behavior symptom.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the dosage of the continuous administration of the dopamine agonist is from about 0.1 mg/kg/day to about 500 mg/kg/day, preferably from about 1 mg/kg/day to about 100 mg/kg/day, and more preferably from about 1 mg/kg/day to about 10 mg/kg/day.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the period of the continuous administration of the dopamine agonist is preferably from about at least 12 hours to about 48 hours, preferably from about at least 12 hours to about 24 hours, and for an oral extended release formulation more preferably about 24 hours by once daily administration.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the continuous administration of the dopamine agonist comprises administration of a combination of the dopamine agonist with one or more other dopamine agonists. The dopamine agonists used in this method include, but are not limited to, Pramipexole, rotigotine, pardoprunox, sumanirole and piribedil.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the continuous administration of the dopamine agonist has an effect of sustained decrease of extracellular level of dopamine.

In another embodiment, the present invention provides methods of treating early morning akinesia, which comprises continuous administration to a patient in need of such treatment a therapeutically effective amount of a dopamine agonist or a pharmaceutically acceptable salt, enantiomer, solvate, hydrate, polymorph or prodrug thereof where the continuous administration of the dopamine agonist has an effect of maintaining a sufficient and stable extracellular level of the dopamine agonist in the striatum of the patient. The phrase “a sufficient and stable extracellular level of the dopamine agonist” as used herein means a level of dopamine agonist that is sufficient to provide a therapeutic effect in treating early morning akinesia over the course of the therapy.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

### Experimental Details

### Materials and Methods

### Animals

The present study was conducted in male Wistar rats (RjHan: W I, Janvier, Le Genest St Isle, France). The animals were housed under a 12 hour light/dark cycle (lights on 06:00-18:00) in temperature (23±2° C.) and humidity (55±5%) controlled rooms with free access to food (GLP Vitamin fortified, Provimi Kliba AG, Kaiseraugst, Switzerland) and water throughout the experiment. All in vivo studies were approved by the appropriate institutional governmental agency (Regierungspraesidium Tuebingen, Germany) and performed in an AAALAC (Association for Assessment and Accreditation of Laboratory Animal Care International)—accredited facility in accordance with the European Convention for Animal Care and Use of Laboratory Animals.

### Haloperidol-Induced Catalepsy

Haloperidol-induced catalepsy is used as an animal model of extrapyramidal side-effects and for screening anti-parkinsonian drugs. Haloperidol is able to induce parkinsonian-like symptoms such as muscle rigidity and catalepsy. Haloperidol-induced catalepsy is considered as an animal model of parkinsonian akinesia which reflects the exaggerated reflex reaction necessary to maintain postural stability, the obstruction to actively challenge stable static equilibrium and to initiate phasic locomotor movements. Haloperidol-induced akinesia is a result of the blockade of DA D₂ receptors in the corpus striatum. Cataleptic immobility is regarded as an animal equivalent of akinesia and is demonstrated by an animal allowing its body to be placed in and maintain abnormal or unusual postures (Sanberg et al., The catalepsy test: its ups and downs. Behay. Neurosci. 102:748-759, 1988; Schmidt et al., Behavioural pharmacology of glutamate in the basal ganglia. J. Neural. Transm. Suppl. 38:65-89 1992).

Catalepsy was induced by treatment of rats with haloperidol (0.5 mg/kg, i.p.) and maintained for 12 hours by administration of haloperidol (0.1 mg/kg, i.p.) every 4 hours. The rats were placed with their forelimbs on a horizontal bar elevated 6 cm from the floor. The time (s) during which the rats maintain in this unusual position was recorded up to 60 seconds (cut-off period 60 seconds). Catalepsy considered fulfilled when the rat moved its forelimbs and stepped down the bar or climbed upon the bar. Three treatment groups were chosen. In the PPX-CR group (n=9), ALZET® osmotic minipumps (model 2004 or 1007D, DURECT Corporation, Cupertino, Calif., USA) filled with PPX solution were implanted subcutaneously under isoflurane anaesthesia the day before the catalepsy experiment. PPX was delivered continuously at the dose of 1 mg/kg/day. The PPX-IR group (n=9) was treated with PPX (1 mg/kg, s.c.) 3 times (morning, midday, evening) on the day before the catalepsy experiment. On the day of the experiment, the first measurement of catalepsy was performed 2 hours after the bolus injection of haloperidol. Subsequently, the PPX-IR and vehicle group (n=9) were treated with PPX (1 mg/kg, s.c.) and vehicle, respectively. Catalepsy was measured 2, 4, 6, 8, 10 and 12 hours later.

### Reserpine-Induced Akinesia

Reserpine-induced akinesia was measured in the open field system Actimot™ (TSE Systems GmbH, Bad Homburg, Germany) for 1 hour in the morning. Rats were placed individually in the centre of the activity box (46.5 cm×46.5 cm) and horizontal motor activity (m) was determined in 10 minutes intervals by infrared sensor pairs (interspace 1.4 cm) with a sampling rate of 100 Hz. Three treatment groups were chosen. In the PPX-CR group (n=7), ALZET® osmotic minipumps (model 1007D, DURECT Corporation, Cupertino, Calif., USA) filled with PPX solution were implanted subcutaneously under isoflurane anaesthesia 3 days before the measurement of akinesia. PPX was delivered continuously at the dose of 2 mg/kg/day. The PPX-IR (n=6) group was treated with PPX (0.3 mg/kg, s.c.) 3 times (morning, midday, evening) on the day before the akinesia measurement. All rats were treated with reserpine (1 mg/kg, s.c.) in the afternoon the day before the experiment. Reserpine was first dissolved in 100% acetic acid and in a subsequent step diluted with water to a final concentration of 1% acetic acid. Seventeen hours later, motor activity was measured in the open field system for 60 minutes (early morning akinesia).

Reserpine acts presynaptically by blocking the uptake of monoamines by the vesicular monoamine transporter (VMAT-2). This inhibition unselectively affects the storage of monoamine neurotransmitters such as adrenaline, noradrenaline, dopamine, histamine and 5-HT in the CNS and also in the periphery. Although not specific to a single neurotransmitter pathway and without involvement of neurodegenerative events, the reserpine model is still a valuable tool to investigate symptomatic effects of dopamine receptor agonists in Parkinson's Disease as well as to study non-dopaminergic mechanisms in Parkinson's Disease. The present study optimised the typical reserpine model by reducing the dose of reserpine to 1 mg/kg.

### In Vivo Microdialysis Surgery

Rats were anaesthetised with a mixture of ketamine (70 mg/kg, i.p.) and xylazine (6 mg/kg, i.p.) and mounted in a stereotaxic frame (David Kopf, Tujunga, Calif., USA) on a flat-skull position. Anaesthesia was maintained by using 0.2-2% isoflurane in N₂O/O₂ (70:30). An intracerebral guide cannula was implanted aiming at the striatum (MAB 4.9.IC, Microbiotech, Stockholm, Sweden) at the following coordinates relative to the bregma: AP: +0.7 mm, ML: +3.0 mm, DV: −3.0 mm (from skull), according to the rat brain atlas of (Paxinos et al., The rat brain in stereotaxic coordinates, Academic Press, San Diego, 1998). A hole was drilled for the placement of the guide cannula, which was fixed to the skull with two stainless steel screws and dental cement (PermaCem, DMG Chemisch-Pharmazeutische Fabrik GmbH, Hamburg, Germany). Subsequently, the ALZET® osmotic minipump (model 1007D, DURECT Corporation, Cupertino, Calif., USA) filled with PPX solution was implanted subcutaneously in rats of the PPX-CR group. PPX was delivered continuously at a dose of 1 mg/kg/day. Following surgery, rats were housed individually in perspex cages and allowed to recover for 2 days before performing the in vivo microdialysis procedure.

### In Vivo Microdialysis Procedure

On the day of the experiment, concentric microdialysis probes (MAB 4.9.4.Cu, 4 mm cuprophane membrane length, Microbiotech, Sweden) were introduced into the guide cannulae and the rats were placed into a microdialysis system with a balanced arm for freely moving animals. The probes were perfused with artificial cerebrospinal fluid (aCSF) containing 147 mM NaCl, 2.7 mM KCl, 1.2 mM CaCl₂, 0.85 mM MgCl₂ and 1 mM Na₂HPO₄, pH 7.0-7.4, at a constant flow rate of 2 μl/min After an equilibration period of 2 hours, dialysis samples were collected every 30 minutes into a vial containing 10 μl of hydrochloric acid (0.1 M). During the night, the sampling interval was prolonged to 60 minutes (20 μl of hydrochloric acid). Fractions 1 to 4 (0-2 h) were used for calculation of the basal levels. After 2 hours, the PPX-IR and PPX-CR group were treated with PPX (0.3 mg/kg, s.c. (n=4)) and vehicle (saline, s.c. (n=4)), respectively. The sampling was then continued for 17.5 hours up to the next morning. The reported data were not corrected for the in vitro recovery, which was 12-14% for DA and 8% for PPX. After the experiments the localisation of the probes was verified and only the rats with appropriate probe placement were included in the experiment.

### HPLC Analysis of Microdialysis Samples

Microdialysis samples were split for high performance liquid chromatography (HPLC) using electrochemical detection (ECD) (60 μl) and liquid chromatography coupled to tandem mass spectrometry (LC-MS/MS) (10 μl) analysis. Samples were analyzed for DA using HPLC-ECD under isocratic conditions. The HPLC system consisted of an ASI-100T autosampler and P680 ISO isocratic pump system (Dionex, Idstein, Germany). The detector potential was set at +650 mV using a glassy carbon electrode and an ISAAC Ag/AgCl reference electrode (Antec VT-03, Leyden, The Netherlands). Chromatographic separation was achieved using a reversed-phase column (Grom-Sil 1200DS-4 HE, 250×4.0 mm id, 5 μm particles, Grace Davison Discovery Sciences, Deerfield, Ill., USA) at 35° C. The mobile phase consisted of 1.85 mM 1-octanesulfonic acid sodium salt, 0.13 mM Na₂EDTA×2H₂O, 8.00 mM NaCl, 57.51 mM NaH₂PO₄, adjusted to pH 2.50 with H₃PO₄, filtered through a 0.22 μm filter, mixed up with 5% acetonitrile and was delivered at a flow rate of 1 ml/min. Aliquots were injected by an autosampler with a cooling module set at 4° C. Data were calculated using an external five-point standard calibration.

### LC-MS/MS Analysis of Microdialysis Samples

Microdialysis samples were analysed for PPX using LC-MS/MS. The LC-MS/MS system comprised an HTS PAL autosampler (CTC Analytics AG, Zwingen, Switzerland), an Agilent 1200 Binary Pump, an Agilent 1200 Micro Vacuum Degasser and an Agilent 1200 Thermostatted Column Compartment (Agilent Technologies, Morges, Switzerland). Mobile phase “A” and “B” consisted of 0.1% formic acid in LC-MS grade water and acetonitrile, respectively. The gradient was chosen as follows: 0.00 min: 100% A, 1.40 min 100% A, 1.41 min 0% A, 2.00 min 0% A, 2.10 min 100% A, 2.50 min 100% A and delivered at 0.5 ml/min onto a reversed-phase column (Synergi Polar-RP 80 A, 150×2.0 mm i. d., 5 μm particles, Phenomenex, Inc., Aschaffenburg, Germany) at 20° C. The column switching valve was set at 0.00 min to the waste, at 0.75 min to the mass spectrometer and at 2.00 min to the waste again. Eluates were detected using an API 4000™ triple quadrupole LC/MS/MS mass spectrometer (MDS Sciex, Ontario, Canada) in the positive electrospray ionisation mode. The ion spray voltage was set at 4500 V and the source temperature at 500° C. Three transitions were chosen: 212-153 (declustering potential (DP) 56 V, collision energy (CE) 21 V, cell exit potential (CXP) 10 V), 212-111 (DP 56 V, CE 37 V, CXP 8 V), 212-126 (DP 56 V, CE 39 V, CXP 8 V) and transition 212-153 was used for the quantification of PPX. As internal standard [D₇]-PPX was analysed using transition 219-153 (DP 86 V, CE 21 V, CXP 10 V).

### Materials

All drugs were calculated as free base. PPX dihydrochloride as well as [D₇]-PPX dihydrochloride was synthesised at Boehringer Ingelheim Pharma GmbH & Co. KG. HPLC and LC-MS/MS chemicals of the highest available purity were obtained from Sigma-Aldrich Chemie GmbH (Steinheim, Germany).

### Statistical Analysis

Statistical analysis was carried out using GraphPad Prism version 5.01 for Windows (GraphPad software, La Jolla, Calif., USA). All values are expressed as mean±SEM. P<0.05 was considered as statistically significant. The time course of haloperidol-induced catalepsy test as well as reserpine-induced akinesia was analysed by a two-way analysis of variance (ANOVA) with treatment as independent and time as dependent factor followed by a Bonferroni post hoc test. Statistical analysis of the cumulative data of the haloperidol-induced catalepsy and the reserpine-induced akinesia test was carried out using one-way ANOVA with treatment as independent factor followed by a Bonferroni post hoc test. For comparison of basal DA and PPX levels an unpaired t-test was performed.

### Results

### Haloperidol-Induced Catalepsy:

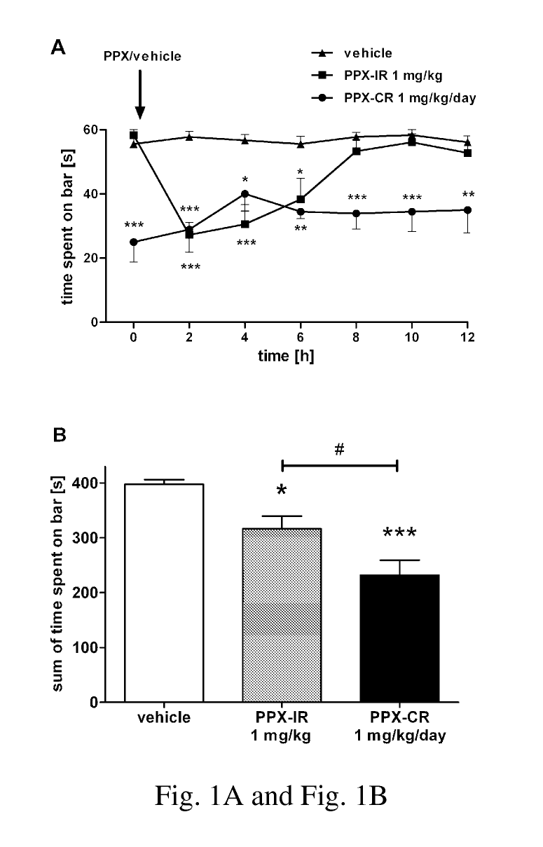

FIG. 1 shows the effect of PPX on haloperidol-induced catalepsy. Specifically, it shows effects of PPX-IR (1 mg/kg, s.c., n=9), PPX-CR (1 mg/kg/day, s.c., n=9) and vehicle (n=9, s.c.) on the time course (FIG. 1A) and cumulative data (FIG. 1B) of haloperidol-induced catalepsy. The PPX-IR group was treated with PPX 3 times on the day before the catalepsy experiment. Haloperidol (0.5 mg/kg, i.p.) was injected 2 hours prior to the first catalepsy measurement. Catalepsy was maintained for 12 hours by administration of haloperidol (0.1 mg/kg, i.p.). Data are expressed as mean±SEM. The time course was analysed by a two-way ANOVA followed by a Bonferroni post hoc test (***P<0.001, **P<0.01, *P<0.05 vs. vehicle). Cumulative data was analysed using one-way ANOVA followed by a Bonferroni post hoc test (***P<0.001, *P<0.05 vs. vehicle, #P<0.05 PPX-IR vs. PPX-CR). Statistical analysis yielded a significant interaction of time×treatment (F(12; 144)=6.388; P<0.001) as well as significant effects on time (F(6; 144)=4.786; P<0.001) and treatment (F(2; 144)=15.33; P<0.001) (FIG. 1A). Time spent on the bar of the PPX-CR group was significantly decreased in comparison to the vehicle group during the whole experiment (Oh, 2 h, 8 h, 10 h: P<0.001; 4 h: P<0.05; 6 h, 12 h: P<0.01). The PPX-IR and vehicle group did not display a significant difference 2 h after haloperidol injection (time point 0; pre-test before PPX/vehicle injection) indicating that pre-treatment with PPX the day before did not show an effect on haloperidol-induced catalepsy the next morning. Following injection with PPX, the time spent on the bar decreased in the PPX-IR group at time points 2 h and 4 h (P<0.001) as well as 6 h (P<0.05). Regarding the cumulative data (FIG. 1B), PPX-CR (P<0.001) as well as PPX-IR (P<0.05) showed an improvement of haloperidol-induced catalepsy, whilst PPX-CR revealed a significant higher effect in comparison to PPX-IR (P<0.05).

The above results indicate that the effects of PPX are dependent upon the PPX exposure in the brain. In particular, the day following acute PPX pre-treatment the symptomatic effects of PPX were no longer present which resulted in early morning akinesia. In contrast, continuous PPX exposure using subcutaneously implanted Alzet® minipumps prevented early morning akinesia. Additionally, continuous PPX exposure produced significantly lower extracellular dopamine levels than the peak decrease obtained after acute PPX administration, although the PPX exposure was lower in the PPX-CR group. The results show a pronounced effect of PPX-IR which was reversible, declined after 6 hours and was absent at 8 hours. Because the effect of a single haloperidol injection led only to significant catalepsy within 6 hours, the catalepsy model was adapted to maintain catalepsy over a longer period of time by multiple haloperidol injections using low haloperidol doses. This adaptation allowed to study the effects of PPX-IR and PPX-CR over a long observation period of 12 hours. It was found that only PPX-CR antagonised the haloperidol-induced catalepsy over the whole observation period which is in agreement with PPX exposure measurements, so the duration of the anti-cataleptic effect was longer in the PPX-CR group than the PPX-IR group.

### Reserpine-Induced Akinesia

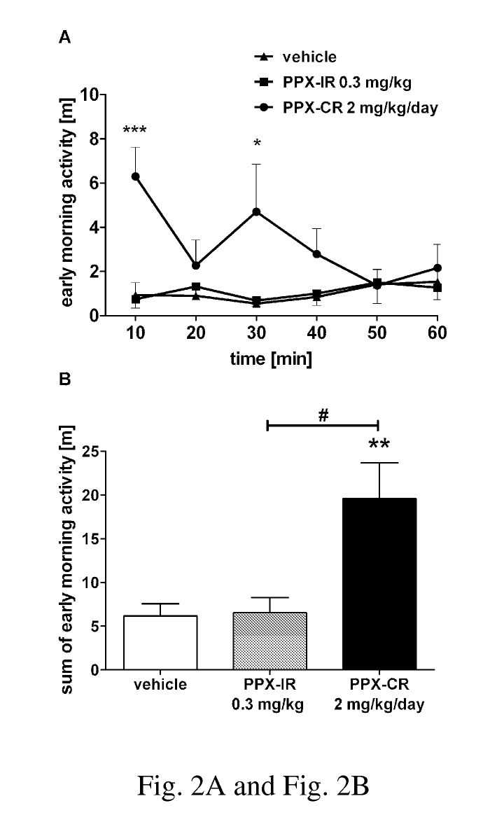

The effects of PPX on reserpine-induced early morning akinesia are shown in FIG. 2. Specifically, FIG. 2 shows effects of PPX-IR (2 mg/kg, s.c., n=7), PPX-CR (2 mg/kg/day, s.c., n=6) and vehicle (n=6, s.c.) on the time course (FIG. 2A) and cumulative data (FIG. 2B) of reserpine-induced akinesia. The PPX-IR group was treated with PPX 3 times on the day before the akinesia measurement. Reserpine (1 mg/kg, s.c.) was injected 17 hours prior to the experiment. Data are expressed as mean±SEM. The time course was analysed by a two-way ANOVA followed by a Bonferroni post hoc test (***P<0.001, *P<0.05 vs. vehicle). The cumulative data was analysed using one-way ANOVA followed by a Bonferroni post hoc test (**P<0.01 vs. vehicle, #P<0.05 PPX-IR vs. PPX-CR). Statistical analysis revealed a significant treatment effect (F(2; 18)=7.266; P<0.01). No significant differences were observed between the vehicle and the PPX-IR group indicating that pre-treatment with PPX the day before does not alter early morning akinesia. In contrast, akinesia was improved by treatment with PPX-CR at 10 min (P<0.001) and 30 min (P<0.05) (FIG. 2A) as well as considering the whole experiment over 60 min (P<0.01) (FIG. 2B). Results from this animal model also indicate that the effects of PPX are dependent upon the PPX exposure in the brain. In this animal model, reserpine was used to investigate the effects of PPX-IR and PPX-CR on the motor behaviour symptom akinesia. Similar to what is observed in the haloperidol-induced catalepsy model, PPX-CR antagonised the motor impairment. PPX-CR was effective over the whole observation period including the first measurement on early morning akinesia.

### Measurement of Extracellular Dopamine Levels

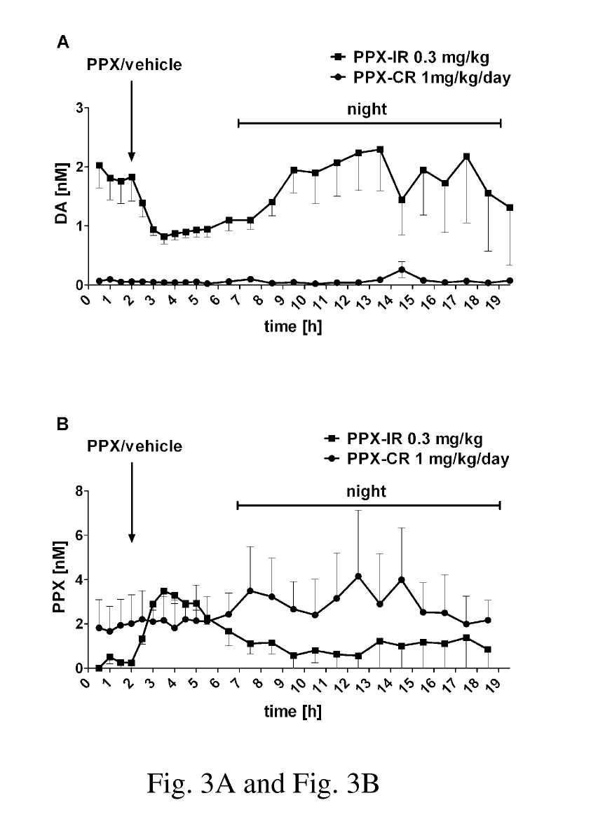

Effects of PPX on extracellular Dopamine levels in the striatum are displayed in FIG. 3A. Pre-dose basal levels of Dopamine in the PPX-IR were found to be 1.86 nM. In the PPX-CR group no pre-dose values could be measured because the microdialysis surgery and the implantation of the pump were carried out at the same time. At the time of Dopamine measurement in the PPX-CR, stable levels of approximately 0.07 nM were obtained which did not vary over time implicating steady state conditions. Statistical analysis revealed significantly lower Dopamine levels in the PPX-CR group (96.2%) in comparison to the pre-dose values of the PPX-IR group (P<0.01). The maximum effect in the PPX-IR group was observed 90 minutes after PPX treatment (44.4% in comparison to basal Dopamine levels). The reduction of extracellular Dopamine levels can be explained by stimulation of presynaptic Dopamine receptors in dopaminergic nerve terminals. This effect is characteristic for Dopamine receptor agonists including PPX and reflects both the involvement on regulation of Dopamine synthesis and on Ca²⁺ dependent exocytotic Dopamine release by the Dopamine autoreceptor mediated feedback inhibition.

### Measurement of Microdialysate PPX Levels

Extracellular PPX levels in the striatum are displayed in FIG. 3B. Pre-dose levels of PPX in the PPX-IR were found to be 0.25 nM. As mentioned above, no pre-dose levels in the PPX-CR were obtained and basal PPX levels of the PPX-CR were found to be 1.86 nM. Injection of PPX in the PPX-IR group led to an increase in PPX levels, which was maximum 90 min following PPX injection (3.48 nM).

Blood Plasma Levels Achieved with Once-Daily Extended Release PPX Formulation

FIG. 4 shows the average blood plasma levels of pramipexole over 24 hours in healthy adult men in fasted state that is built up and maintained by the once daily application of an extended release formulation wherein pramipexole is the active ingredient. (See also FIG. 4 of Peter Jenner, M. Könen_Bergmann, C. Schepers, S. Härtter, Clinical Therapeutics 31 (11) 2009 2968-2711). Such blood plasma levels of pramipexole over 24 hours were achieved using extended release tablet formulations according to Table 1 above at five different dosage levels (0.375 mg, 0.75 mg, 1.5 mg. 3.0 mg and 4.5 mg).

1. A method for treating early morning akinesia, comprising continuous administration to a patient in need of such treatment a therapeutically effective amount of a dopamine agonist or a pharmaceutically acceptable salt, enantiomer, solvate, hydrate, polymorph or prodrug thereof.

2. The method according to claim 1, wherein the continuous administration of the dopamine agonist has an effect of continuous dopaminergic stimulation.

3. The method according to claim 1, wherein the continuous administration of the dopamine agonist is achieved by subcutaneous infusion of the dopamine agonist.

4. The method according to claim 3, wherein the subcutaneous infusion of the dopamine agonist is achieved by one or more subcutaneously implanted minipumps.

5. The method according to claim 1, wherein the continuous administration of the dopamine agonist is achieved by oral administration of a sustained or extended release formulation where the dopamine agonist is an active ingredient in the formulation.

6. The method according to claim 1, wherein the patient has Parkinson's Disease.

7. The method according to claim 1, wherein the dopamine agonist is a nonergot dopamine agonist.

8. The method according to claim 7, wherein the nonergot dopamine agonist is pramipexole or a pharmaceutically acceptable salt thereof.

9. The method according to claim 1, wherein such treatment reduces or eliminates one or more symptoms, diseases or conditions associated with or resulting from early morning akinesia.

10. The method according to claim 9, wherein the symptom, disease or condition associated with or resulting from early morning akinesia is catalepsy.

11. The method according to claim 9, wherein the symptom, disease or condition associated with or resulting from early morning akinesia is muscle rigidity.

12. The method according to claim 9, wherein the symptom, disease or condition associated with or resulting from early morning akinesia is motor behavior symptom.

13. The method according to claim 1, wherein the dosage of the continuous administration of the dopamine agonist is from about 0.1 mg/kg/day to about 500 mg/kg/day.

14. The method according to claim 1, wherein the dosage of the continuous administration of the dopamine agonist is from about 1 mg/kg/day to about 100 mg/kg/day.

15. The method according to claim 1, wherein the dosage of the continuous administration of the dopamine agonist is from about 1 mg/kg/day to about 10 mg/kg/day.

16. The method according to claim 1, wherein the period of the continuous administration of the dopamine agonist is from about 0.1 to about 48 hours.

17. The method according to claim 1, wherein the period of the continuous administration of the dopamine agonist is from about 1 to about 24 hours.

18. The method according to claim 1, wherein the period of the continuous administration of the dopamine agonist is from about 1 to about 12 hours.

19. The method according to claim 1, wherein the continuous administration of the dopamine agonist comprises administering a combination of the dopamine agonist with one or more other dopamine agonists.

20. The method according to claim 19, wherein the one or more other dopamine agonists are rotigotine, pardoprunox, sumanirole or piribedil.

21. The method according to claim 1, wherein the continuous administration of the dopamine agonist has an effect of sustained decrease of extracellular level of dopamine.

22. The method according to claim 1, wherein the continuous administration of the dopamine agonist has an effect of maintaining a sufficient and stable extracellular level of the dopamine agonist in the striatum of the patient.

23. The method according to claim 5, wherein the oral sustained or extended release formulation is administered once every 12 hours, once every 24 hours, once every 36 hours or once every 48 hours at a predetermined time of the day.

24. The method according to claim 5, wherein the oral sustained or extended release formulation is administered once every 24 hours at a predetermined time of the day.

25. The method according to claim 5, wherein the oral sustained or extended release formulation is administered once every 24 hours in the evening prior to the patient going to sleep.
